(19) United States
(12) Patent Application Publication
(10) Pub. No.: US 2025/0090132 A1
(43) Pub. Date: Mar. 20, 2025

(54) BLOOD FLOW MEASURING DEVICE

(71) Applicant: EDGECARE INC., Seoul (KR)

(72) Inventors: Jung Jun KIM, Seoul (KR); II Seob SONG, Goyang-si (KR); Doo Young GO, Seoul (KR)

(73) Assignee: EDGECARE INC., Seoul (KR)

(21) Appl. No.: 18/680,545

(22) Filed: May 31, 2024

(30) Foreign Application Priority Data
Sep. 19, 2023 (KR) 10-2023-0124482

(51) Int. Cl. A61B 8/06; A61B 8/00; A61B 8/08

(52) U.S. Cl. CPC A61B 8/06; A61B 8/4488; A61B 8/4494; A61B 8/488

(57) ABSTRACT

A blood flow measuring device according to an embodiment of the present disclosure includes a linear array, a phased array, and a calculation unit. The linear array is divided into a plurality of sub-arrays, each of the plurality of sub-arrays transmitting a transmission ultrasonic signal to an object along a line corresponding to each of the plurality of sub-arrays. The phased array receives a reception ultrasonic signal obtained by reflecting the transmission ultrasonic signal from the object. The calculation unit calculates an energy for a doppler signal in a blood vessel region included in the object based on the reception ultrasonic signal.

### CROSS-REFERENCE TO RELATED APPLICATION(S)

This application claims priority under 35 U.S.C. § 119 to Korean Patent Application No. 10-2023-0124482, filed Sep. 19, 2023, the content of which is incorporated by reference herein in its entirety.

### BACKGROUND

### Field

The present disclosure relates to a blood flow measuring device.

### Description of the Related Art

When a blood flow rate is measured using an ultrasonic image, it may be difficult to accurately measure a blood flow rate due to a human movement. In order to solve this problem, various studies have recently been conducted.

### SUMMARY

The present disclosure provides a blood flow measuring device capable of overcoming difficulty caused by a human movement in measuring an energy for a doppler signal in a blood vessel region, by calculating an energy for a doppler signal in a blood vessel region included in an object, with a transmission ultrasonic signal being transmitted, using a linear array divided into a plurality of sub-arrays, from each of the plurality of sub-arrays to the object along a line corresponding to each of the plurality of sub-arrays, and a reception ultrasonic signal reflected from the object being received using a phased array.

According to an embodiment of the present disclosure, a blood flow measuring device includes a linear array, a phased array, and a calculation unit. The linear array is divided into a plurality of sub-arrays, each of the plurality of sub-arrays transmitting a transmission ultrasonic signal to an object along a line corresponding to each of the plurality of sub-arrays. The phased array receives a reception ultrasonic signal obtained by reflecting the transmission ultrasonic signal from the object. The calculation unit calculates an energy for a doppler signal in a blood vessel region included in the object based on the reception ultrasonic signal.

The plurality of sub-arrays may simultaneously transmit the transmission ultrasonic signals to the object along the lines corresponding to the plurality of sub-arrays, respectively, at a first time.

After the first time, a selected sub-array corresponding to the line crossing the blood vessel region among the plurality of sub-arrays may transmit the transmission ultrasonic signal to the object at every predetermined time interval.

When the energy for the doppler signal calculated from the calculation unit is smaller than a predetermined reference energy, the plurality of sub-arrays may simultaneously transmit the respective transmission ultrasonic signals to the object.

The blood flow measuring device may further include a control unit. The control unit may divide the selected sub-array into a plurality of partial arrays and control the plurality of partial arrays.

The control unit may simultaneously transmit the transmission ultrasonic signals to the blood vessel region along partial lines corresponding to the partial arrays at a second time after the first time.

The calculation unit may select a selected partial array, which is a partial array corresponding to a selected partial line corresponding to the highest energy among the energies for the doppler signals corresponding to the respective partial lines.

The control unit may drive the selected partial array at every predetermined time interval after the second time to transmit the transmission ultrasonic signal to the blood vessel region.

The linear array may transmit an image ultrasonic transmission signal to the object and receive an image ultrasonic reception signal reflected from the object. The blood vessel region may be identified according to an ultrasonic image generated from an imaging unit based on the image ultrasonic reception signal. The control unit may select the selected sub-array among the plurality of sub-arrays corresponding to the blood vessel region to transmit the transmission ultrasonic signal.

The phased array may be disposed in a first direction or in a second direction that is a direction opposite to the first direction with respect to the linear array.

In addition to the technical aspects of the present disclosure discussed above, other features and advantages of the present disclosure will be set forth below, or may be apparent to those skilled in the art to which the present disclosure pertains from the following description.

According to the present disclosure, the following effect may be obtained.

In the blood flow measuring device according to the present disclosure, by calculating an energy for a doppler signal in a blood vessel region included in the object, with a transmission ultrasonic signal being transmitted, using the linear array divided into the plurality of sub-arrays, from each of the plurality of sub-arrays to the object along the line corresponding to each of the plurality of sub-arrays, and a reception ultrasonic signal reflected from the object being received using the phased array, it is possible to overcome difficulty caused by a human movement in measuring an energy for a doppler signal in the blood vessel region.

Further, other features and advantages of the present disclosure may be understood through the embodiments of the present disclosure.

### BRIEF DESCRIPTION OF THE DRAWINGS

The above and/or other aspects of the disclosure will be more apparent by describing certain embodiments of the disclosure with reference to the accompanying drawings, in which:

FIG. 1 is a diagram illustrating a blood flow measuring device according to embodiments of the present disclosure;

FIG. 2 is a diagram for explaining an operation of the blood flow measuring device of FIG. 1;

FIG. 3 is a diagram for explaining one embodiment of the blood flow measuring device of FIG. 1;

FIG. 4 is a diagram for explaining an operation of a control unit included in the blood flow measuring device of FIG. 1;

FIGS. 5 and 6 are diagrams for explaining another embodiment of the blood flow measuring device of FIG. 1;

FIG. 7 is a diagram for explaining an operation example of a linear array included in the blood flow measuring device of FIG. 1; and

FIG. 8 is a diagram for explaining how to arrange a linear array and a phased array included in the blood flow measuring device of FIG. 1.

### DETAILED DESCRIPTION

In the specification, in adding reference numerals for elements throughout the drawings, it should be noted that like reference numerals are used to denote like elements, wherever possible, even though the elements are shown in different drawings.

The terms used in the specification should be understood as follows.

Singular expressions should be understood to include plural expressions unless the context clearly indicates otherwise, and the scope should not be limited by the terms.

It should further be understood that the terms “include”, “have”, and the like do not preclude the presence or addition of one or more other features, integers, steps, operations, elements, parts, or combinations thereof.

Hereinafter, preferred embodiments of the present disclosure designed to solve the aforementioned problem will be described in detail with reference to the accompanying drawings.

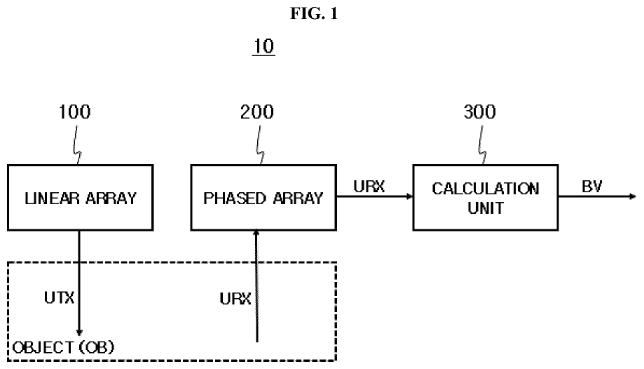

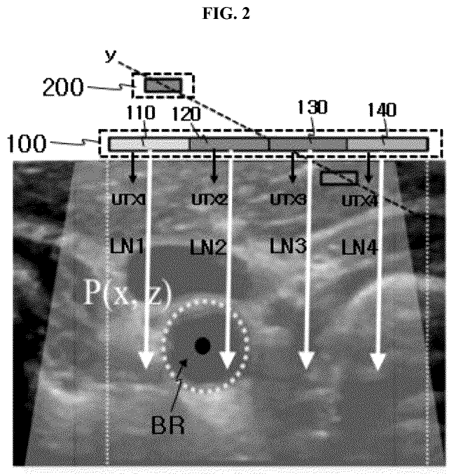

FIG. 1 is a diagram illustrating a blood flow measuring device according to embodiments of the present disclosure, and FIG. 2 is a diagram for explaining an operation of the blood flow measuring device of FIG. 1.

Referring to FIGS. 1 and 2, a blood flow measuring device 10 according to an embodiment of the present disclosure may include a linear array 100, a phased array 200, and a calculation unit 300. The linear array 100 can be divided into a plurality of sub-arrays. For example, the plurality of sub-arrays may include a first sub-array 110, a second sub-array 120, a third sub-array 130, and a fourth sub-array 140. If the linear array 100 consists of 64 elements, each of the first sub-array 110 to the fourth sub-array 140 may consist of 16 elements. Here, the plurality of sub-arrays are four sub-arrays, but the plurality of sub-arrays according to the present disclosure is not limited thereto and may be embodied in various ways.

Each of the plurality of sub-arrays may transmit a transmission ultrasonic signal (UTX) to an object OB along a line corresponding to each of the plurality of sub-arrays. For example, the plurality of sub-arrays may include a first sub-array 110 to a fourth sub-array 140. The line arranged vertically with respect to the first sub-array 110 in a direction toward the object OB may be a first line LN1, and the line arranged vertically with respect to the second sub-array 120 in a direction toward the object OB may be a second line LN2. In addition, the line arranged vertically with respect to the third sub-array 130 in a direction toward the object OB may be a third line LN3, and the line vertically with respect to the fourth sub-array 140 arranged in a direction toward the object OB may be a fourth line LN4.

In this case, the first sub-array 110 may transmit a first transmission ultrasonic signal UTX1 toward the first line LN1, and the second sub-array 120 may transmit a second transmission ultrasonic signal UTX2 toward the second line LN2. In addition, the third sub-array 130 may transmit a third transmission ultrasonic signal UTX3 toward the third line LN3, and the fourth sub-array 140 may transmit a fourth transmission ultrasonic signal UTX4 toward the fourth line LN4.

Thereafter, the phased array 200 may receive a reception ultrasonic signal URX obtained by reflecting the transmission ultrasonic signal UTX from the object OB. For example, the first transmission ultrasonic signal UTX1 to the fourth transmission ultrasonic signal UTX4 transmitted from the first sub-array 110 to the fourth sub-array 140 to the object OB may be reflected from the object OB and received as a reception ultrasonic signal URX by the phased array 200.

The calculation unit 300 may calculate an energy BV for a doppler signal in a blood vessel region BR included in the object OB based on the reception ultrasonic signal URX. For example, by processing the reception ultrasonic signal URX, the calculation unit 300 may identify a blood vessel region BR included in the object OB, and calculate an energy BV for a doppler signal in the blood vessel region BR. The blood vessel region BR may be a region containing blood vessels in a human body.

In the blood flow measuring device 10 according to the present disclosure, by calculating an energy BV for a doppler signal in a blood vessel region BR included in the object OB with a transmission ultrasonic signal UTX being transmitted, using the linear array 100 divided into the plurality of sub-arrays, from each of the plurality of sub-arrays to the object OB along the line corresponding to each of the plurality of sub-arrays, and a reception ultrasonic signal URX reflected from the object OB being received using the phased array 200, it is possible to overcome difficulty caused by a human movement in measuring an energy BV for a doppler signal.

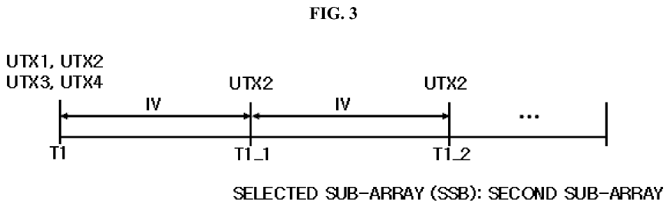

FIG. 3 is a diagram for explaining one embodiment of the blood flow measuring device of FIG. 1.

Referring to FIGS. 1 to 3, in one embodiment, the plurality of sub-arrays may simultaneously transmit transmission ultrasonic signals UTX to the object OB along the lines corresponding to the plurality of sub-arrays, respectively, at a first time T1. For example, the plurality of sub-arrays may include a first sub-array 110 to a fourth sub-array 140. At the first time T1, the first sub-array 110 to the fourth sub-array 140 may simultaneously transmit the first transmission ultrasonic signal UTX1 to the fourth transmission ultrasonic signal UTX4 to the object OB along the first line LN1 to the fourth line LN4.

In one embodiment, after the first time T1, a selected sub-array SSB corresponding to a line crossing the blood vessel region BR among the plurality of sub-arrays may transmit a transmission ultrasonic signal UTX to the object OB at every predetermined time interval IV. For example, after the first time T1, the phased array 200 may receive a reception ultrasonic signal URX and provide the reception ultrasonic signal URX to the calculation unit 300. The calculation unit 300 may determine a blood vessel region BR by processing the reception ultrasonic signal URX. In this case, the line crossing the blood vessel region BR among the first line LN1 to the fourth line LN4 may be the second line LN2, and the sub-array corresponding to the second line LN2 may be the second sub-array 120. Here, the selected sub-array SSB may be the second sub-array 120.

In a case where the selected sub-array SSB is the second sub-array 120, the blood flow measuring device 10 according to the present disclosure may transmit a transmission ultrasonic signal UTX to the blood vessel region BR using the second sub-array 120 at every predetermined time interval IV after the first time T1 to calculate an energy BV for a doppler signal in the blood vessel region.

In one embodiment, when the energy BV for the doppler signal calculated from the calculation unit 300 is smaller than a predetermined reference energy, the plurality of sub-arrays may simultaneously transmit respective transmission ultrasonic signals UTX to the object OB. For example, while the blood flow measuring device 10 according to the present disclosure transmits a transmission ultrasonic signal UTX to the blood vessel region BR using the second sub-array 120 at every predetermined time interval IV after the first time T, the energy BV for the doppler signal may be smaller than the predetermined reference energy. In this case, the first sub-array 110 to the fourth sub-array 140 may simultaneously transmit a first transmission ultrasonic signal UTX1 to a fourth transmission ultrasonic signal UTX4 to the object OB along the first line LN1 to the fourth line LN4. Thereafter, based on a reception ultrasonic signal URX received by the phased array 200, the calculation unit 300 may calculate an energy BV for a doppler signal, and detect again a selected sub-array SSB corresponding to a line crossing the blood vessel region BR.

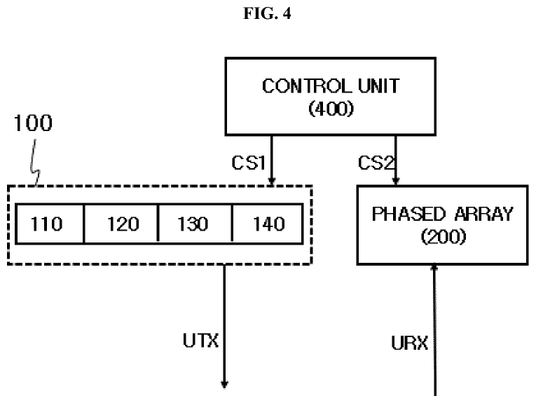

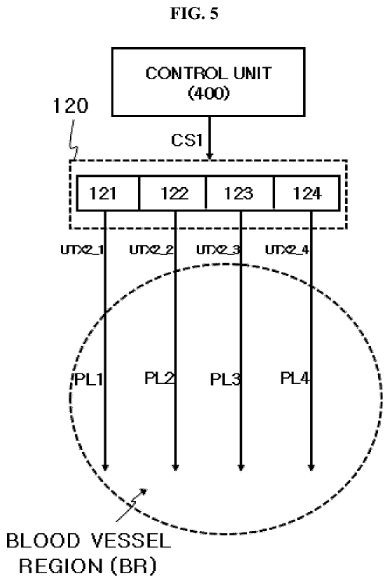

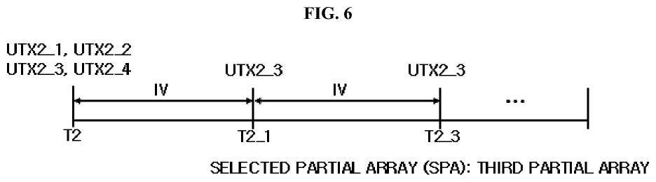

FIG. 4 is a diagram for explaining an operation of a control unit included in the blood flow measuring device of FIG. 1, and FIGS. 5 and 6 are diagrams for explaining another embodiment of the blood flow measuring device of FIG. 1.

Referring to FIGS. 1 to 6, in one embodiment, the blood flow measuring device 10 may further include a control unit 400. The control unit 400 can divide the selected sub-array SSB into a plurality of partial arrays and control the plurality of partial arrays. For example, the control unit 400 may control the operations of the linear array 100, the phased array 200, and the blood flow device using control signals, and the control signals may include a first control signal CS1 and a second control signal CS2. The first control signal CS1 may be used to control the linear array 100, and the second control signal CS2 may be used to control the phased array 200.

For example, the linear array 100 may include a first sub-array 110 to a fourth sub-array 140. The selected sub-array SSB among the first sub-array 110 to the fourth sub-array 140 may be the second sub-array 120, and the control unit 400 may further divide the second sub-array 120 corresponding to the selected sub-array SSB into a plurality of partial arrays. Here, the plurality of partial arrays may include a first partial array 121 to a fourth partial array 124. The control unit 400 may control the first to fourth partial arrays 121 to 124 using the first control signal CS1.

In one embodiment, the control unit 400 may simultaneously transmit transmission ultrasonic signals UTX to the blood vessel region BR along partial lines corresponding to the partial arrays at a second time T2 after the first time T1. For example, the plurality of partial arrays may include a first partial array 121 to a fourth partial array 124. The line arranged vertically with respect to the first partial array 121 in a direction toward object OB may be a first partial line PL1, and the line arranged vertically with respect to the second partial array 122 in a direction toward object OB may be a second partial line PL2. In addition, the line arranged vertically with respect to the third partial array 123 in a direction toward object OB may be a third partial line PL3, and the line arranged vertically with respect the fourth partial array 124 in a direction toward object OB may be a fourth partial line PL4.

In this case, at the second time T2 after the first time T1, the first partial array 121 to the fourth partial array 124 may simultaneously transmit a 2_1st transmission ultrasonic signals UTX2_1 to a 2_4th transmission ultrasonic signal UTX2_4 to the blood vessel region BR included in the object OB along the first partial line PL1 to the fourth partial line PL4.

In one embodiment, the calculation unit 300 may select a selected partial array SPA, which is a partial array corresponding to a selected partial line corresponding to the highest energy among energies BV for doppler signals corresponding to the respective partial lines. For example, after the second time T2, the phased array 200 may receive a reception ultrasonic signal URX and provide the reception ultrasonic signal URX to the calculation unit 300. By processing the reception ultrasonic signal URX, the calculation unit 300 may compare a first energy BV1 in an area where the first partial line PL1 and the blood vessel region BR overlap each other, a second energy BV2 in an area where the second partial line PL2 and the blood vessel region BR overlap each other, a third energy BV3 in an area where the third partial line PL3 and the blood vessel region BR overlap each other, and a fourth energy BV4 in an area where the fourth partial line PL4 and the blood vessel region BR overlap each other, calculate the third energy BV3 as the highest energy BV, and select the third partial array 123 corresponding to the third energy BV3 as a selected partial array SPA.

In one embodiment, the control unit 400 may drive the selected partial array SPA at every predetermined time interval IV after the second time T2 to transmit a transmission ultrasonic signal UTX to the blood vessel region BR. For example, in a case where the selected partial array SPA is the third partial array 123, the blood flow measuring device 10 according to the present disclosure may calculate an energy BV for a doppler signal by transmitting a transmission ultrasonic signal UTX to the blood vessel region BR using the third partial array 123 at every predetermined time interval IV after the second time T2.

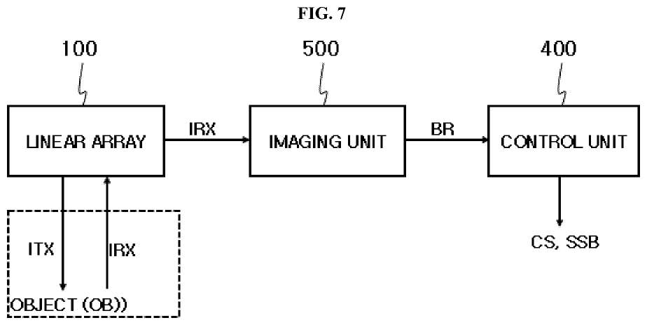

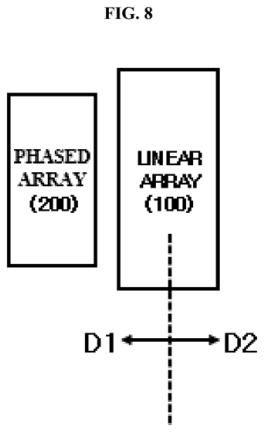

FIG. 7 is a diagram for explaining an operation example of the linear array included in the blood flow measuring device of FIG. 1, and FIG. 8 is a diagram for explaining how to arrange the linear array and the phased array included in the blood flow measuring device of FIG. 1.

Referring to FIGS. 1 to 8, in one embodiment, the linear array 100 may transmit an image ultrasonic transmission signal ITX to the object OB and receive an image ultrasonic reception signal IRX reflected from the object OB. A blood vessel region BR can be identified according to an ultrasonic image generated by an imaging unit 500 based on the image ultrasonic reception signal. The control unit 400 may transmit a transmission ultrasonic signal UTX by selecting a selected sub-array SSB among the plurality of sub-arrays corresponding to the blood vessel region BR. For example, as another operation example of the blood flow measuring device 10 according to the present disclosure, a blood vessel region BR may be detected through an ultrasonic image generated by transmitting and receiving an image ultrasonic signal using the linear array 100, and the control unit 400 may transmit a transmission ultrasonic signal UTX to the blood vessel region BR through the selected sub-array SSB corresponding to the blood vessel region BR.

In one embodiment, the phased array 200 may be arranged in a first direction D1 or in a second direction D2 that is a direction opposite to the first direction D1 with respect to the linear array 100. For example, the first direction D1 may be a leftward direction with respect to the linear array 100, and the second direction D2 may be a rightward direction with respect to the linear array 100.

In the blood flow measuring device 10 according to the present disclosure, by calculating an energy BV for a doppler signal in a blood vessel region BR included in the object OB, with a transmission ultrasonic signal UTX being transmitted, using the linear array 100 divided into the plurality of sub-arrays, from each of the plurality of sub-arrays to the object OB along the line corresponding to each of the plurality of sub-arrays, and a reception ultrasonic signal URX reflected from the object OB being received using the phased array 200, it is possible to overcome difficulty caused by a human movement in measuring an energy BV for a doppler signal.

The present disclosure discloses a method of transmitting a beam through multiple sub-apertures, but the present disclosure is not limited thereto. The present disclosure may be implemented in such a manner that a broad beam is transmitted by entirely or partially applying a delay value to the linear array 100 and acquiring blood flow data through data received from the phased array 200. The broad beam may be an ultrasonic signal that is transmitted by focusing on a certain area in a direction toward the elements at the focusing depth using all or some of the elements included in the linear array, rather than focusing on one point, so that energy is uniformly transmitted to the certain area. The certain area may be smaller or wider than the linear array range.

What is claimed is:

1. A blood flow measuring device comprising:
a linear array divided into a plurality of sub-arrays, each of the plurality of sub-arrays transmitting a transmission ultrasonic signal to an object along a line corresponding to each of the plurality of sub-arrays;
a phased array receiving a reception ultrasonic signal obtained by reflecting the transmission ultrasonic signal from the object; and
a calculation unit calculating an energy for a doppler signal in a blood vessel region included in the object based on the reception ultrasonic signal.

2. The blood flow measuring device of claim 1, wherein the plurality of sub-arrays simultaneously transmit the transmission ultrasonic signals to the object along the lines corresponding to the plurality of sub-arrays, respectively, at a first time.

3. The blood flow measuring device of claim 2, wherein after the first time, a selected sub-array corresponding to the line crossing the blood vessel region among the plurality of sub-arrays transmits the transmission ultrasonic signal to the object at every predetermined time interval.

4. The blood flow measuring device of claim 3, wherein when the energy for the doppler signal calculated from the calculation unit is smaller than a predetermined reference energy, the plurality of sub-arrays simultaneously transmits the respective transmission ultrasonic signals to the object.

5. The blood flow measuring device of claim 4, further comprising:
a control unit dividing the selected sub-array into a plurality of partial arrays and controlling the plurality of partial arrays.

6. The blood flow measuring device of claim 5, wherein the control unit simultaneously transmits the transmission ultrasonic signals to the blood vessel region along partial lines corresponding to the partial arrays at a second time after the first time.

7. The blood flow measuring device of claim 6, wherein the calculation unit selects a selected partial array, which is a partial array corresponding to a selected partial line corresponding to the highest energy among the energies for the doppler signals corresponding to the respective partial lines.

8. The blood flow measuring device of claim 7, wherein the control unit drives the selected partial array at every predetermined time interval after the second time to transmit the transmission ultrasonic signal to the blood vessel region.

9. The blood flow measuring device of claim 8, wherein
the linear array transmits an image ultrasonic transmission signal to the object and receives an image ultrasonic reception signal reflected from the object,
the blood vessel region is identified according to an ultrasonic image generated from an imaging unit based on the image ultrasonic reception signal, and
the control unit selects the selected sub-array among the plurality of sub-arrays corresponding to the blood vessel region to transmit the transmission ultrasonic signal.

10. The blood flow measuring device of claim 9, wherein the phased array is disposed in a first direction or in a second direction that is a direction opposite to the first direction with respect to the linear array.

11. The blood flow measuring device of claim 1, wherein the transmission ultrasonic signal is a broad beam generated using all or some of elements included in the linear array.
